(19) United States
(12) Patent Application Publication
(10) Pub. No.: US 2025/0255994 A1
(43) Pub. Date: Aug. 14, 2025

(54) DIMERIC IMMUNOCONJUGATES FOR USE IN TREATING CANCERS AND METHODS OF USE

(71) Applicant: Medicovestor, Inc., Wilmington, DE (US)

(72) Inventor: Seah Lim, Wilmington, DE (US)

(21) Appl. No.: 18/964,025

(22) Filed: Nov. 29, 2024

Related U.S. Application Data
(60) Provisional application No. 63/604,155, filed on Nov. 29, 2023.

(51) Int. Cl. A61K 51/10; A61K 47/68; A61P 35/00; C07K 16/30

(52) U.S. Cl. CPC A61K 51/1093; A61K 47/68037; A61K 47/6809; A61K 47/6869; A61K 51/1072; A61P 35/00; C07K 16/3069; A61K 2121/00

(57) ABSTRACT

This disclosure relates to dimeric immunoconjugates for use in treating cancer generally and to dimeric immunoconjugates that bind human sperm protein 17 (Sp17) specifically. A dimeric immunoconjugate of this disclosure generally comprises a first monomeric antibody that carries a first payload and a second monomeric antibody that carries a second payload, wherein the first payload and the second payload are different. Such dimeric immunoconjugates advantageously allow the simultaneous delivery of two chemotherapeutics to a cancer cell, which allows for synergistic antineoplastic activity.

### CROSS REFERENCE TO RELATED APPLICATION

This is an application claims the benefit of U.S. Patent Application No. 63/604,155, filed 29 Nov. 2023, the disclosure of which is incorporated herein, in its entirety, by this reference.

### SEQUENCE LISTING

This disclosure includes a sequence listing, which has file name “1200590030_Sequence_Listing.xml,” which was created on Mar. 3, 2025, which has a file size of 13,995 bytes, and which is incorporated by reference in its entirety.

### FIELD OF THE INVENTION

The present disclosure relates to dimeric immunoconjugates for use in treating cancer generally and to dimeric immunoconjugates that bind human sperm protein 17 (Sp17) specifically.

### BACKGROUND

Therapeutic antibodies are responsible for tremendous improvements in cancer outcomes and present new opportunities to cure cancer, at least in a subset of patients. The first antibody cancer immunotherapeutic Rituxan® was approved to treat B-cell non-Hodgkin's lymphoma in the United States in 1997 and has over $100 billion in lifetime sales. Rituxan® still sells over $1 billion annually despite extensive competition. The competing antibody Zevalin®, for example, was approved in the United States in 2002. Both Rituxan® and Zevalin® target CD20, which is a B-cell antigen, and both immunotherapeutics act by depleting B cells. When Rituxan® binds CD20, it triggers antibody-dependent cellular toxicity and leukocyte-mediated cell death, whereas Zevalin® is chemically modified to chelate a radioisotope, which additionally allows for radiation-induced cell death. Therapeutic antibodies may also be conjugated to cytotoxic pharmaceuticals, for example, with labile linkers that allow antibody-drug conjugates that release their cytotoxic payloads upon binding to an antigen. Numerous other antibody-based strategies exist as cancer treatments.

While therapeutic antibodies revolutionized the field of oncology, progress remains incremental. Cancer is not yet cured. Innovative strategies that improve upon existing antibody technologies remain desirable.

### SUMMARY

Various aspects of this disclosure relate to the discovery of advantages provided by dimeric immunoconjugates that improve upon the efficacies of conventional monomeric immunoconjugates and naked immunotherapeutics. The dimeric immunoconjugates of this disclosure advantageously carry a different payload on each of their two constituent monomers such that targeting a single cancer cell with a dimeric immunoconjugate allows for the simultaneous delivery of two different payloads to the cancer cell, which results in synergistic antineoplastic activity. Dimeric immunoconjugates also generally comprise two Fc fragments, which allow the targeting of cancer cells by leukocytes such as natural killer cells, and can therefore simultaneously deliver a third, independent mechanism to neutralize a cancer cell. Dimeric immunoconjugates may also mediate complement-mediated lysis of the target cells more efficiently.

Dimeric immunoconjugates that comprise two different payloads may be prepared, for example, by crosslinking a first immunoconjugate that comprises a first payload with a second immunoconjugate that comprises a second payload.

Dimeric immunoconjugates display pharmacokinetics, pharmacodynamics, and avidity that vary from their constituent monomers. These factors combine to both favorably alter the administration and efficacy of dimeric immunoconjugates in unpredictable ways that were not previously appreciated. The inventors unexpectedly discovered that, while simultaneous administration of two different monomeric immunoconjugates can display synergy, crosslinking two different monomeric immunoconjugates to produce a dimeric immunoconjugate displays enhanced synergy. Two different monomeric immunoconjugates may have different pharmacodynamics and/or pharmacokinetics, for example, such that simultaneous administration does not correspond with simultaneous binding to the same cancer cell, and random variation in antigen-binding events and/or endocytosis means that two different monomeric immunoconjugates may fail to simultaneously deliver their payloads to the same cancer cell even when pharmacodynamics and pharmacokinetics are similar. Dimeric immunoconjugates of this disclosure improve upon the foregoing two issues at least because dimeric immunoconjugates nearly always deliver two payloads simultaneously. Additionally, certain cancer-associated antigens display low cell-surface expression, low accessibility to antibodies, or other confounding features that reduce the probability that multiple immunotherapeutics will bind to the same cancer cell. The dimeric immunoconjugates of this disclosure are particularly effective at targeting such antigens because (1) dimeric immunoconjugates generally carry twice the payload as monomeric immunoconjugates, and thus, a single binding event delivers twice the payload, and (2) only a single binding event is required to deliver a synergistic combination of different payloads.

Without limiting this disclosure or any patent claim that matures from this disclosure, the inventors have found that dimeric immunoconjugates that bind cancer antigens are more favorable than conventional immunoconjugates because dimeric immunoconjugates can cross-link cancer cells, which can inhibit metastases that spread cancer to other parts of the body and can also inhibit the escape of the cancer from a localized intervention or immune response.

Conventional, naked therapeutic antibodies generally induce Fc-receptor-mediated cell death, and conventional immunoconjugates allow for both Fc-receptor-mediated cell death and antineoplastic activity based upon their payloads. The payloads, however, risk masking the Fc portion of an antibody, which risks limiting Fc-receptor-mediated activity. Dimeric immunotherapeutics generally further mask the Fc portions of an antibody and further limit Fc-receptor-based antineoplastic activity. Impaired Fc-receptor-mediated cytotoxicity might be expected to reduce the efficacy of a dimeric immunoconjugate, but the inventors found the opposite. Dimeric immunoconjugates display improved efficacy relative to monomeric immunoconjugates, which was unexpected, for example, due to the compromised Fc-receptor-mediated cytotoxicity of a dimeric immunoconjugate.

Without limiting this disclosure or any patent claim that matures from this disclosure, the inventors found that limiting Fc-receptor-based antineoplastic activity can be leveraged to reduce non-specific Fc binding and improve safety relative to conventional immunotherapeutics. Such advantages may be especially useful in the reticuloendothelial system including the liver by reducing the nonspecific side-effects of an antibody-drug conjugate. Elimination of the dependence of an immunoconjugate upon a supplementary Fc-receptor pathway therefore allows the development of immunoconjugates with greater drug-to-antibody ratios and also allows the administration of higher doses of a dimeric immunoconjugate while minimizing the risks posed by non-specific Fc-related toxicity.

Without limiting this disclosure or any patent claim that matures from this disclosure, the inventors found that dimeric immunoconjugates that bind cancer antigens are more favorable than conventional immunoconjugates, for example, because additional binding sites favor clustering of cell-surface antigens and resultant endocytosis, which sequesters the immunoconjugate inside a cancer cell to favor antineoplastic efficacy. Sequestering an immunoconjugate that comprises a radioisotope within a cancer cell both accelerates cell death and protects other cells from the radiation. Many immunoconjugates that comprise pharmaceutical agents include labile linkers that are engineered to release the pharmaceutical agents upon endocytosis, for example, in response to the pH of an endocytic vesicle or in response to a hydrolase or other enzyme of a vesicle. Dimeric immunoconjugates can accelerate release of pharmaceutical agents that become labile upon endocytosis by favoring clustering that results in endocytosis.

Another unexpected advantage of dimeric immunoconjugates is that one, two, or each of their improved efficacy, pharmacokinetics, and pharmacodynamics allow for reduced infusion times relative to monomeric immunoconjugates. Therapeutic antibodies used as cancer immunotherapeutics are frequently infused intravenously over a period of time such as several hours. As the development of therapeutic antibodies and other infused therapeutic interventions increases, the capacity of infusion centers has become limiting. Pharmacological interventions that can be administered over shorter periods of time relative to comparable interventions can therefore reduce the burden on infusion centers, reduce costs, and improve patient experiences independent from the improved clinical outcomes described herein.

Without limiting this disclosure or any patent claim that matures from this disclosure, dimeric immunoconjugates may also improve the cytotoxicity of cancer cells by increasing the number of immunoconjugates that bind to the cancer cells since it is possible that not all of the binding domains of an immunoconjugate making up a dimeric immunoconjugate will be used when binding an antigen. This may overcome some of the obstacles encountered in targeting antigens that display low copy numbers on cancer cells by increasing the number of the constituent immunoconjugates of a dimeric immunoconjugate in proximity to and/or engaged with the cancer cells. Dimeric immunoconjugates may, therefore, be ideal for targeting cancers that display a low expression density of a cell surface antigen. Dimeric immunoconjugates are also about twice the size of a monomeric immunoconjugate and can therefore carry about twice the payload as monomeric immunoconjugates to increase potency. A single antigen-binding event therefore positions twice as much payload in proximity to a target cell as a monomeric immunoconjugate.

The following disclosure broadly describes various other novel aspects of dimeric immunoconjugates for oncological uses. Nothing in this summary section, the preceding background and field sections, or the following brief description of the drawings, detailed description, and exemplification sections shall limit any patent claim that matures from this disclosure; any such patent claim shall instead be construed according to the claim language itself in the context of its claim dependency.

### BRIEF DESCRIPTION OF THE DRAWINGS

FIG. 1 is an image of an SDS-PAGE gel loaded with a molecular weight standard (lane M) and 2 micrograms of recombinant Sp17 protein under reducing conditions (lane 1) and non-reducing conditions (lane 2).

FIG. 2 consists of two panels. The left panel is a Coomassie-blue stained SDS-PAGE gel loaded with a molecular weight standard (lane M), the anti-Sp17 antibody chAB2 under reducing conditions (lane 1), and the anti-Sp17 antibody chAB2 under non-reducing conditions (lane 2). The right panel is a western blot of an SDS-PAGE gel loaded with a molecular weight standard (lane M) and the anti-Sp17 antibody chAB2 under reducing conditions (lane E), in which chAB2 was detected using enhanced chemiluminescence (ECL) with anti-mouse secondary antibodies.

FIG. 3 is a western blot of an SDS-PAGE gel loaded with a molecular weight standard (lane M) and with Sp17 protein (lanes 1, 2, & 3), in which the Sp17 protein was detected with the anti-Sp17 antibody chAB2.

FIG. 4 consists of three panels. The left panel is a graph that depicts flow cytometry results for ID8 cells incubated with an anti-mouse fluorescein isothiocyanate (FITC) antibody, but without a primary antibody. The middle panel is a graph that depicts flow cytometry results for ID8 cells labeled with the anti-Sp17 antibody chAB2 and the anti-mouse FITC antibody. The right panel is a histogram that displays an increased mean fluorescent intensity for the ID8 cells labeled with chAB2 and the anti-mouse FITC antibody of the middle panel relative to ID8 cells incubated with the anti-mouse FITC antibody alone of the left panel.

FIG. 5 is a high-performance liquid chromatography (HPLC) chromatogram that demonstrates that anti-Sp17 antibody chAB2 preparations can be manufactured at 93 percent purity.

FIG. 6 is a graph that displays surface plasmon resonance data for the anti-Sp17 antibody chAB2 binding to a recombinant 6His-Sp17 protein. The lines correspond, from highest to lowest, to fitted cycles for 300 nanomolar, 100 nanomolar, 50 nanomolar, 25 nanomolar, 12.5 nanomolar, and 6.25 nanomolar concentrations of 6His-Sp17. This data was used to calculate an association rate constant (ka) for chAB2 to Sp17 of 62,600 per mole per second, a dissociation rate constant (kd) of 0.000 129 8 per second, and a dissociation constant (KD) of 2.073 nanomolar.

FIG. 7 is an image of an SDS-PAGE gel loaded with a molecular weight standard (lane M) and the anti-Sp17 antibody SP17-AB2 under reducing conditions (lane 1) and non-reducing conditions (lane 2).

FIG. 8 is a western blot of an SDS-PAGE gel loaded with a molecular weight standard (lane M), with Sp17 protein (lane 1), and with a tumor cell lysate (lane 2), in which the Sp17 protein was detected with the anti-Sp17 antibody SP17-AB2.

FIGS. 9A, 9B, and 9C are graphs that depict immunotherapeutic concentration (x-axis, nanomolar) versus cytotoxicity against SK-OV-3 cells (y-axis, percent cytotoxicity) for immunotherapeutics selected from the monomeric antibody SP17-AB2 (circles, ●), the monomeric immunoconjugate SP17-AB2-DXD (squares, ▪), a control human IgG4 antibody (upright triangles, ▴), and deruxtecan (inverted triangles, ▾).

FIG. 10 is a graph that depicts immunotherapeutic concentration (x-axis, nanomolar) versus cytotoxicity against ID8 cells (y-axis, percent cytotoxicity) for immunotherapeutics selected from the monomeric antibody SP17-AB2 (circles, ●), the immunoconjugate SP17-AB2-DXD (squares, ▪), hIgG4 (upright triangles, ▴), and deruxtecan (inverted triangles, ▾).

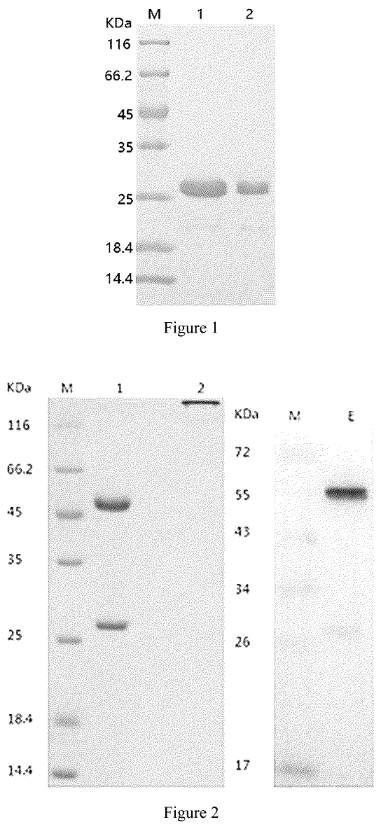

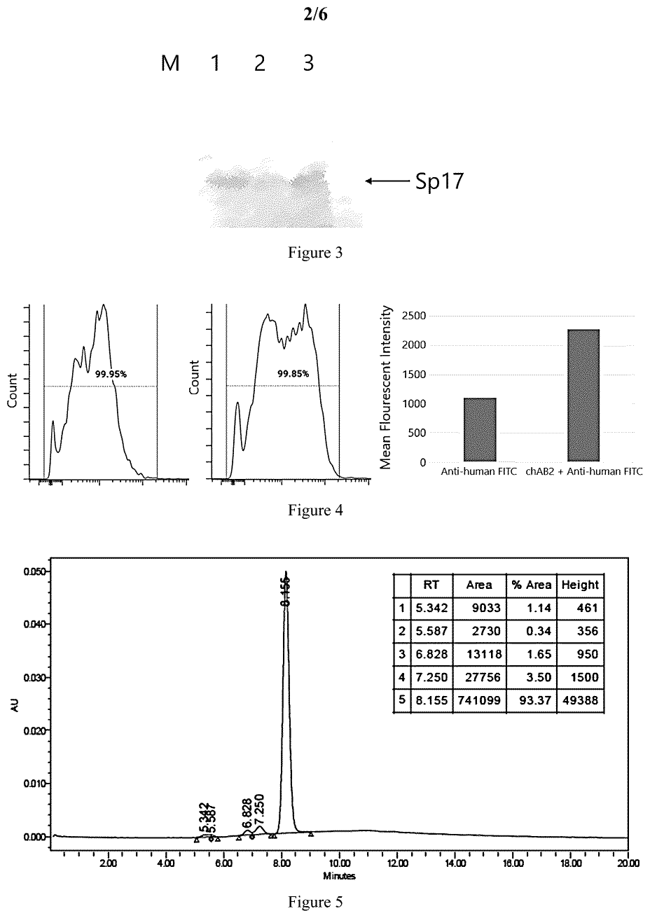

### DETAILED DESCRIPTION

Various aspects of this disclosure relate to a method to treat cancer in a human subject, comprising administering a therapeutically effective amount of an immunotherapeutic to the subject. The term “immunotherapeutic,” as the term is used in this disclosure without additional context, refers to dimeric immunoconjugates. The immunotherapeutic generally comprises two covalently-crosslinked antibodies that are each conjugated to a different payload.

Various aspects of this disclosure relate to an immunotherapeutic, comprising a first immunoconjugate and a second immunoconjugate that are covalently crosslinked.

In this disclosure, an “immunoconjugate” is an antibody that is conjugated to either a pharmaceutical agent or a radioisotope. The precise conjugation strategy is not limiting. An immunoconjugate that is conjugated to a pharmaceutical agent is nevertheless typically covalently crosslinked to the pharmaceutical agent with a linker as described infra. An immunoconjugate that is conjugated to a radioisotope is typically covalently crosslinked to a chelating agent, which chelating agent chelates the radioisotope as described infra.

The term “antibody” includes immunoglobulins (Ig's) of different classes (for example, IgA, IgG, IgM, IgD, and IgE) and subclasses (for example, IgG2a and IgG4) and includes fully-human antibodies, chimeric antibodies, and engineered variants thereof. This specification describes, for example, a chimeric human-mouse IgG (chAB2) as well as a fully-human IgG (SP17-AB2) that was engineered to contain a Ser228Pro mutation. The dimeric immunoconjugates of this disclosure typically comprise IgG antibodies, but the precise class of an immunoglobulin is not limiting.

Many strategies exist for preparing dimeric immunoconjugates, and the precise linking strategy shall not limit this disclosure or any claim that matures from this disclosure. A first monomeric immunoconjugate may be combined, for example, with a molar excess of sulfosuccinimidyl 4-(N-maleimidomethyl)cyclohexane-1-carboxylate (sulfo-SMCC) or succinimidyl 4-(N-maleimidomethyl)cyclohexane-1-carboxylate (SMCC) to modify one or more amines of the immunoconjugate (such as an epsilon-amino group of a lysine) with a thiol-reactive maleimide. This conjugation strategy is generally compatible with antibodies and is used, for example, to crosslink mertansine to trastuzumab to create the antibody-drug conjugate trastuzumab emtansine, which is also known as Kadcyla®. A second monomeric immunoconjugate may be combined, for example, with a molar excess of N-succinimidyl S-acetylthiopropionate (SATP) or N-succinimidyl S-acetylthioacetate (SATA), for example, to modify one or more amines of the immunoconjugate (such as an epsilon-amino group of a lysine) with an acetyl-protected thiol. The thiol may then be deprotected, for example, under acidic conditions with hydroxylamine. An approximately equimolar amount of the first monomeric immunoconjugate and second monomeric immunoconjugate may then be crosslinked. The resultant dimeric immunoconjugate may be purified, for example, by dialysis and/or size-exclusion chromatography.

Antibodies contain dozens of lysine residues, and thus, chemical crosslinking based upon a strategy that modifies primarily epsilon-amino groups of lysines will generally result in a heterogenous mixture of various different dimers that display various different efficacies. Precise quaternary structure may be enforced, for example, by cloning cysteines into specific positions within an antibody to add thiols that can covalently crosslink two different antibodies with a disulfide bond. Such strategies are known and described, for example, in Shopes, B., “A genetically engineered human IgG mutant with enhanced cytolytic activity,” J. IMMUNOL. 1992 148 (9): 2918-22. Cloning thiols into antibodies to create a disulfide bond that crosslinks the monomeric constituents of a dimeric immunoconjugate can also advantageously preserve the epsilon-amino groups of lysines of the monomeric constituents for conjugation to payloads.

In this disclosure, the term “cancer” refers to carcinomas, sarcomas, lymphomas, leukemias, germ cell tumors, and blastomas of any type. In some embodiments, the cancer is brain cancer, ovarian cancer, breast cancer, vaginal cancer, vulvar cancer, uterine cancer, cervical cancer, endometrial cancer, prostate cancer, testicular cancer, penile cancer, liver cancer, intrahepatic bile duct cancer, lung cancer, small cell lung cancer, non-small cell lung cancer, bronchial cancer, mesothelioma, pancreatic cancer, gall bladder cancer, non-melanoma skin cancer, melanoma, Kaposi sarcoma, thyroid cancer, head and neck cancer, nasopharyngeal cancer, oropharyngeal cancer, hypopharyngeal cancer, laryngeal cancer, oral cavity cancer, tongue cancer, mouth cancer, salivary gland cancer, esophageal cancer, gastric cancer, colorectal cancer, colon cancer, rectal cancer, anal cancer, kidney cancer, renal cell cancer, renal pelvis cancer, bladder cancer, urethral cancer, Hodgkin lymphoma, non-Hodgkin's lymphoma, myeloma, multiple myeloma, acute lymphocytic leukemia, chronic lymphocytic leukemia, acute myeloid leukemia, chronic myeloid leukemia, osteosarcoma, or soft tissue cancer. In some specific embodiments, the cancer is multiple myeloma, ovarian cancer, or non-small cell lung cancer.

In this disclosure, the term “treat” refers to at least one of: to cure a cancer; to increase the probability that a cancer will be cured; to shorten the time over which a cancer is cured; to increase the probability that the time necessary to cure a cancer will be shortened; to decrease the severity of a cancer; to increase the probability that the severity of a cancer will decrease; to shorten the time over which the severity of a cancer is decreased; to increase the probability that the time necessary to decrease the severity of a cancer will be shortened; to inhibit a cancer from worsening; to increase the probability that a cancer will not worsen; to delay the worsening of a cancer; to increase the probability that the worsening of a cancer will be delayed; to inhibit the occurrence or recurrence of a cancer; to decrease the probability that a cancer will occur or reoccur; to delay the onset of a cancer; to increase the probability that the onset of a cancer will be delayed; to alleviate at least one symptom of a cancer; to increase the probability that at least one symptom of a cancer will be alleviated; to shorten the time over which at least one symptom of a cancer is alleviated; to increase the probability that the time necessary to alleviate at least one symptom of a cancer will be shortened; to decrease the severity of at least one symptom of a cancer; to increase the probability that the severity of at least one symptom of a cancer will be decreased; to shorten the time over which the severity of at least one symptom of a cancer is decreased; to increase the probability that the time necessary to decrease the severity of at least one symptom of a cancer will be shortened; to inhibit at least one symptom of a cancer from worsening; to increase the probability that at least one symptom of a cancer will not worsen; to delay the worsening of at least one symptom of a cancer; to increase the probability that the worsening of at least one symptom of a cancer will be delayed; to inhibit at least one symptom of a cancer from occurring or reoccurring; to decrease the probability that at least one symptom of a cancer will occur or reoccur; to delay the onset of at least one symptom of a cancer; and to increase the probability that the onset of at least one symptom of a cancer will be delayed.

In this disclosure, the term “therapeutically effective amount” is an amount that is effective to treat cancer, which amount is generally first-determined empirically based upon pre-clinical data and then confirmed by performing pilot and/or phase I-II clinical trials.

In some embodiments, the administering is performed by injection. In some specific embodiments, the injection is an intravenous injection, intradermal injection, subcutaneous injection, intramuscular injection, intraperitoneal injection, or intratumoral injection. In some very specific embodiments, the injection is an intravenous injection.

An immunotherapeutic of this disclosure generally comprises a first immunoconjugate and a second immunoconjugate that are covalently crosslinked. The first immunoconjugate generally comprises a first antibody that binds to a first cancer-associated antigen and a first payload selected from a first radioactive isotope and a first pharmaceutical agent. The second immunoconjugate generally comprises a second antibody that binds to a second cancer-associated antigen and a second payload selected from a second radioactive isotope and a second pharmaceutical agent. In some embodiments, the first cancer-associated antigen and the second cancer-associated antigen are the same.

The first antibody and the second antibody of an immunotherapeutic are not necessarily identical and may vary, for example, in post-translational modifications that generally result in heterogeneous glycosylation patterns. In some embodiments, the first antibody and the second antibody of an immunotherapeutic are encoded by the same nucleotide sequence(s); such antibodies generally have the same amino acid sequence. In some embodiments, the first antibody and the second antibody of a immunotherapeutic are encoded by different nucleotide sequences that encode different amino acid sequences, which different amino acid sequences may, for example, enforce a specific quaternary structure such as with one or more of inter-IgG disulfide bond(s), create complementary sterics, and/or provide complementary electrostatic charges to facilitate favorable orientations between the first antibody and the second antibody that allow for improved avidity relative to random or otherwise un-optimized orientations.

In some embodiments, the first antibody and the second antibody have amino acid sequences with at least 90 percent sequence homology. In some specific embodiments, the first antibody and the second antibody have amino acid sequences with at least 95 percent sequence homology. In some very specific embodiments, the first antibody and the second antibody have amino acid sequences with at least 98 percent sequence homology.

As used in this disclosure, the term “sequence homology” refers to percent “positives” as determined by Standard Protein BLAST® over the full length of a sequence. Standard Protein BLAST® is available at https://blast.ncbi.nlm.nih.gov/Blast.cgi?PROGRAM-blastp. BLAST® is generally described in Altschul, et al. (1997), “Gapped BLAST and PSI-BLAST: a new generation of protein database search programs”, Nucleic Acids Res. 25:3389-3402, and in Altschul, et al. (2005) “Protein database searches using compositionally adjusted substitution matrices”, FEBS J. 272:5101-5109. As used in this disclosure, the term “sequence identity” refers to the percent of exact matches over the full length of a sequence.

In some embodiments, the first antibody and the second antibody have amino acid sequences with at least 90 percent sequence identity. In some specific embodiments, the first antibody and the second antibody have amino acid sequences with at least 95 percent sequence identity. In some very specific embodiments, the first antibody and the second antibody have amino acid sequences with at least 98 percent sequence identity.

In some embodiments, the first antibody and the second antibody have identical amino acid sequences.

In some embodiments, the immunotherapeutic is a dimer of a first immunoconjugate and a second immunoconjugate of the same antibody. In some specific embodiments, the immunotherapeutic is a dimer of a first immunoconjugate and a second immunoconjugate of an antibody selected from 3F8, abagovomab, abituzumab, adecatumumab, amatuximab, andecaliximab, anrukinzumab, apolizumab, ascrinvacumab, atezolizumab, avelumab, basiliximab, bavituximab, belimumab, bemarituzumab, bermekimab, bevacizumab, bivatuzumab, bleselumab, blontuvetmab, brontictuzumab, cabiralizumab, camrelizumab, capromab, carotuximab, cemiplimab, cetrelimab, cetuximab, cibisatamab, cirmtuzumab, cixutumumab, clazakizumab, codrituzumab, conatumumab, cusatuzumab, dacetuzumab, daclizumab, dalotuzumab, daratumumab, dectrekumab, demcizumab, detumomab, dinutuximab, dinutuximab beta, dostarlimab, drozitumab, duligotuzumab, durvalumab, dusigitumab, ecromeximab, edrecolomab, eldelumab, elgemtumab, elotuzumab, elsilimomab, emactuzumab, emibetuzumab, enavatuzumab, enoblituzumab, enoticumab, ensituximab, epratuzumab, etaracizumab, etigilimab, faricimab, farletuzumab, fibatuzumab, ficlatuzumab, figitumumab, flanvotumab, fresolimumab, futuximab, galiximab, ganitumab, gatipotuzumab, gevokizumab, gilvetmab, gimsilumab, girentuximab, gomiliximab, icrucumab, ifabotuzumab, imalumab, imaprelimab, imgatuzumab, inebilizumab, inolimomab, intetumumab, ipilimumab, iratumumab, isatuximab, iscalimab, istiratumab, labetuzumab, lacnotuzumab, lebrikizumab, lenzilumab, leronlimab, lexatumumab, lintuzumab, lirilumab, lucatumumab, lulizumab pegol, lumiliximab, lumretuzumab, lutikizumab, mapatumumab, margetuximab, matuzumab, milatuzumab, mitumomab, modotuximab, mogamulizumab, monalizumab, namilumab, narnatumab, navicixizumab, naxitamab, necitumumab, nesvacumab, nimotuzumab, nivolumab, obinutuzumab, ocaratuzumab, ocrelizumab, ofatumumab, olaratumab, oleclumab, olokizumab, omburtamab, ontuxizumab, oregovomab, otlertuzumab, panitumumab, pankomab, parsatuzumab, pasotuxizumab, patritumab, pembrolizumab, pemtumomab, pertuzumab, pidilizumab, pritumumab, prolgolimab, racotumomab, radretumab, ramucirumab, ravagalimab, relatlimab, retifanlimab, rilotumumab, rinucumab, rituximab, robatumumab, romilkimab, rosmantuzumab, samalizumab, sarilumab, selicrelumab, seribantumab, sibrotuzumab, siltuximab, sintilimab, sirukumab, spartalizumab, tabalumab, tafasitamab, talacotuzumab, tarextumab, tavolimab, telisotuzumab, tenatumomab, teneliximab, tepoditamab, teprotumumab, theralizumab, tigatuzumab, timigutuzumab, tiragotumab, tislelizumab, tomuzotuximab, tositumomab, tovetumab, tralokinumab, trastuzumab, tremelimumab, ublituximab, ulocuplumab, urelumab, utomilumab, vanalimab, vantictumab, vanucizumab, varisacumab, varlilumab, veltuzumab, volociximab, vonlerolizumab, votumumab, xentuzumab, zalutumumab, zatuximab, zenocutuzumab, and zolbetuximab. Dimeric versions of immunoconjugates of the foregoing antibodies may be manufactured, for example, with the SATP/SATA and sulfo-SMCC/SMCC cross-linking strategies described herein.

In some embodiments, the immunotherapeutic is a dimer of a first immunoconjugate and a second immunoconjugate of a bispecific IgG antibody that comprises two antigen-binding sites that each specifically bind a different cancer-associated antigen such that the immunotherapeutic comprises exactly four antigen-binding sites that each specifically bind two different cancer-associated antigens. In some specific embodiments, the immunotherapeutic comprises two different immunoconjugates of amivantamab, glofitamab, talquetamab, or teclistamab, which are each bispecific antibodies.

In some embodiments, the first cancer-associated antigen and the second cancer-associated antigen are the same.

In some embodiments, the cancer-associated antigen is 4-1BB, 5′-nucleotidase, 5T4, activin receptor-like kinase 1, alpha-fetoprotein, angiopoietin 2, AXL, B7-H3, B-cell activating factor (BAFF), B-cell maturation antigen (BCMA), c-Met, C242, CA-125, CanAg, carbonic anhydrase 9 (CA-IX), carcinoembryonic antigen, CCR4, CCR5, CD3, CD4, CD19, CD20, CD22, CD23, CD25, CD27, CD28, CD30, CD33, CD37, CD38, CD40, CD44, CD51, CD56, CD70, CD74, CD79B, CD80, CD123, CD134, CD152, CD200, CD276, CD319, CEACAM5, claudin 18, coagulation factor III, connective tissue growth factor (CTGF), colony stimulating factor 1 (CSF1), colony stimulating factor 1 receptor (CSF1R), colony stimulating factor 2 (CSF2), CTLA-4, CXCR4, dendritic cell-associated lectin 2, DLL3, DLL4, DR5, EGFL7, EGFR, endoglin, EpCAM, ephrin receptor A3 (EPHA3), epidermal growth factor receptor (EGFR), ERBB3 (HER3), fibroblast activation protein alpha (FAP), FGFR2, fibronectin extra domain-B, folate hydrolase, folate receptor 1, Frizzled receptor, GD2 ganglioside, GD3 ganglioside, gelatinase B, glycoprotein 100 (gp100), glypican 3, GPNMB, G protein-coupled receptor 5D (GPRC5D), GUCY2C, hepatocyte growth factor (HGF), HER1, HER2, HGFR, histone complex, HLA-DR, human scatter factor receptor kinase, IGF-1 receptor (IGF-1R; CD221), IGF-2, interleukin 1 alpha, interleukin-2, interleukin-6, interleukin-13, integrin alph5beta1, integrin alphaVbeta3, KIR2D, LAG3, Lewis-Y antigen, LIV-1, LRRC15, macrophage migration inhibitory factor (MIF), MCP-1, melanoma cell adhesion molecule (MCAM), mesothelin, MUC1, MUC5AC, nectin-4, NGNA ganglioside, Notch 1, Notch receptor, NRP1, PCDC1, PD-1, PD-L1, PDGFRA, phosphate-sodium co-transporter, phosphatidylserine, PTK7, root plate-specific spondin 3, ROR1, SDC1, SLAMF7, SLITRK6, Sp17, STEAP1, TEM1, tenascin C, TGF-beta, TIGIT, TRAIL-R1, TRAIL-R2, tumor-associated calcium signal transducer 2 (TROP-2), tumor antigen CTAA16.88, tumor-specific glycosylated MUC1, tumor-associated glycoprotein 72 (TAG-72), TWEAK receptor, TYRP1, VEGF-A, VEGFR-1, VEGFR-2, or vimentin. In some specific embodiments, the cancer-associated antigen is Sp17.

In some embodiments, the cancer comprises cells that express Sp17.

In some embodiments, the first antibody and the second antibody each comprise two antigen-binding sites that each specifically bind Sp17 such that the immunotherapeutic comprises exactly four antigen-binding sites that each specifically bind Sp17.

In some embodiments, the first antibody and the second antibody each comprise a first variable domain and a second variable, the first variable domain of the first antibody and the first variable domain of the second antibody have identical amino acid sequences, and the second variable domain of the first antibody and the second variable domain of the second antibody have identical amino acid sequences.

In some embodiments, the first variable domain of the first antibody comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7. In some embodiments, the first variable domain of the second antibody comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7. In some specific embodiments, the first variable domain of the first antibody and the first variable domain of the second antibody each comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7.

In some embodiments, the second variable domain of the first antibody comprises a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10. In some embodiments, the second variable domain of the second antibody comprises a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10. In some specific embodiments, the second variable domain of the first antibody and the second variable domain of the second antibody each comprise a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10.

In some embodiments, the first antibody comprises a heavy chain that has at least 90 percent sequence homology with SEQ ID NO: 11 and a light chain that has at least 90 percent sequence homology with SEQ ID NO: 12. In some embodiments, the second antibody comprises a heavy chain that has at least 90 percent sequence homology with SEQ ID NO: 11 and a light chain that has at least 90 percent sequence homology with SEQ ID NO: 12. In some specific embodiments, the first antibody comprises a heavy chain that has at least 90 percent sequence homology with SEQ ID NO: 11 and a light chain that has at least 90 percent sequence homology with SEQ ID NO: 12, and the second antibody comprises a heavy chain that has at least 90 percent sequence homology with SEQ ID NO: 11 and a light chain that has at least 90 percent sequence homology with SEQ ID NO: 12.

In some embodiments, the first antibody comprises a heavy chain that has at least 90 percent sequence identity with SEQ ID NO: 11 and a light chain that has at least 90 percent sequence identity with SEQ ID NO: 12. In some embodiments, the second antibody comprises a heavy chain that has at least 90 percent sequence identity with SEQ ID NO: 11 and a light chain that has at least 90 percent sequence identity with SEQ ID NO: 12. In some specific embodiments, the first antibody comprises a heavy chain that has at least 90 percent sequence identity with SEQ ID NO: 11 and a light chain that has at least 90 percent sequence identity with SEQ ID NO: 12, and the second antibody comprises a heavy chain that has at least 90 percent sequence identity with SEQ ID NO: 11 and a light chain that has at least 90 percent sequence identity with SEQ ID NO: 12.

In some embodiments, the first payload and the second payload are different.

In some embodiments, the first payload is a first radioactive isotope. In some embodiments, the first radioactive isotope is selected from actinium-225, astatine-211, bismuth-212, bismuth-213, copper-67, gallium-68, holmium-166, iodine-124, iodine-131, lutetium-177, samarium-153, technetium-99, terbium-149, and yttrium-90.

In some embodiments, the first radioactive isotope is an alpha emitter. In some specific embodiments, the first radioactive isotope is selected from actinium-225, astatine-211, bismuth-212, bismuth-213, and terbium-149.

In some embodiments, the second payload is a second radioactive isotope. In some embodiments, the second radioactive isotope is selected from actinium-225, astatine-211, bismuth-212, bismuth-213, copper-67, gallium-68, holmium-166, iodine-124, iodine-131, lutetium-177, samarium-153, technetium-99, terbium-149, and yttrium-90.

In some embodiments, the second radioactive isotope is a beta emitter. In some specific embodiments, the second radioactive isotope is selected from copper-67, gallium-68, holmium-166, iodine-124, iodine-131, lutetium-177, technetium-99, samarium-153, and yttrium-90.

In some embodiments, (1) the first payload is a first radioactive isotope, (2) the first radioactive isotope is an alpha emitter, (3) the second payload is a second radioactive isotope, and (4) the second radioactive isotope is a beta emitter.

Examples of antibodies conjugated to radioactive isotopes include tositumomab (also known as BEXXAR®) and ibritumomab tiuxetan (also known as ZEVALIN®). Those of ordinary skill are capable of designing antibodies that are conjugated to radioactive isotopes using known strategies such as those used to conjugate iodine-131 in tositumomab and to conjugate yttrium-90 or indium-111 to ibritumomab (see, for example, U.S. Pat. No. 6,565,827 & 7,422,739, which are incorporated by reference in their entirety). Other examples of antibodies conjugated to radioactive isotopes include clivatuzumab tetraxetan, lilotomab satetraxetan, and tacatuzumab tetraxetan. In some embodiments, the first immunotherapeutic is selected from clivatuzumab tetraxetan, ibritumomab tiuxetan, lilotomab satetraxetan, tacatuzumab tetraxetan, and tositumomab.

Immunotherapeutics of this disclosure may advantageously be prepared, for example, with the same chelator bound to a different radioisotope. A single batch of an antibody can be conjugated, for example, to a tetraxetan moiety that is capable of chelating cations. A first portion of the batch can be used to chelate a first radioactive isotope, a second portion can be used to chelate a second radioactive isotope, and the first portion and the second portion can subsequently be crosslinked to produce the immunotherapeutic. This strategy results in an approximately equal distribution of the first radioactive isotope and the second radioactive isotope on the immunotherapeutics relative, for example, to chelating the first radioactive isotope and the second radioactive isotope simultaneously, which would result in a distribution of individual molecules of an immunotherapeutic that chelate different proportions of the first radioactive isotope and the second radioactive isotope. Using ibritumomab tiuxetan (ZEVALIN®) to chelate two different radioactive isotopes, for example, would result in some molecules of ibritumomab tiuxetan that are enriched in one or another radioactive isotope and some molecules that only chelate a single radioactive isotope. The immunotherapeutics of this disclosure can advantageously avoid such distributions and instead ensure that all molecules chelate both radioactive isotopes.

In some embodiments, (1) the first payload is a first pharmaceutical agent, (2) the second payload is a second pharmaceutical agent, (3) the first pharmaceutical agent and the second pharmaceutical agent are selected from a calicheamicin, camptothecin, deruxtecan, doxorubicin, emtansine, exatecan, irinotecan, maleimidocaproyl monomethyl auristatin F, mertansine, monomethyl auristatin F, paclitaxel, PE38, pyrrolobenzodiazepine, SN-38, and vedotin; and (4) the first pharmaceutical agent and the second pharmaceutical agent are different pharmaceutical agents.

Examples of antibodies conjugated to pharmaceutical agents include gemtuzumab ozogamicin (also known as MYLOTARG®) and trastuzumab emtansine (also known as KADCYLA®). Those of ordinary skill are capable of designing antibodies that are conjugated to pharmaceutical agents using known strategies such as those used to conjugate calicheamicin to gemtuzumab and emtansine to trastuzumab (see, for example, U.S. Pat. No. 5,877,296 & 8,088,387, which are incorporated by reference in their entirety).

Other examples of antibodies conjugated to pharmaceutical agents include anetumab ravtansine, aprutumab ixadotin, azintuxizumab vedotin, belantamab mafodotin, brentuximab vedotin, camidanlumab tesirine, cantuzumab mertansine, cantuzumab ravtansine, cbr96-doxorubicin immunoconjugate, cergutuzumab amunaleukin, cofetuzumab pelidotin, coltuximab ravtansine, denintuzumab mafodotin, depatuxizumab mafodotin, derbyotuximab biotin, enapotamab vedotin, enfortumab vedotin, glembatumumab vedotin, iladatuzumab vedotin, indatuximab ravtansine, indusatumab vedotin, inotuzumab ozogamicin, ladiratuzumab vedotin, laprituximab emtansine, lifastuzumab vedotin, loncastuximab tesirine, lorvotuzumab mertansine, losatuxizumab vedotin, mirvetuximab soravtansine, moxetumomab pasudotox, naratuximab emtansine, pinatuzumab vedotin, polatuzumab vedotin, rovalpituzumab tesirine, sacituzumab govitecan, samrotamab vedotin, sirtratumab vedotin, sofituzumab vedotin, taplitumomab paptox, telisotuzumab vedotin, tisotumab vedotin, trastuzumab deruxtecan, trastuzumab duocarmazine, tucotuzumab celmoleukin, vadastuximab talirine, vandortuzumab vedotin, and vorsetuzumab mafodotin.

In some embodiments, the first immunoconjugate is selected from anetumab ravtansine, aprutumab ixadotin, azintuxizumab vedotin, belantamab mafodotin, brentuximab vedotin, camidanlumab tesirine, cantuzumab mertansine, cantuzumab ravtansine, cbr96-doxorubicin immunoconjugate, cergutuzumab amunaleukin, cofetuzumab pelidotin, coltuximab ravtansine, denintuzumab mafodotin, depatuxizumab mafodotin, derbyotuximab biotin, enapotamab vedotin, enfortumab vedotin, gemtuzumab ozogamicin, glembatumumab vedotin, iladatuzumab vedotin, indatuximab ravtansine, indusatumab vedotin, inotuzumab ozogamicin, ladiratuzumab vedotin, laprituximab emtansine, lifastuzumab vedotin, loncastuximab tesirine, lorvotuzumab mertansine, losatuxizumab vedotin, mirvetuximab soravtansine, moxetumomab pasudotox, naratuximab emtansine, pinatuzumab vedotin, polatuzumab vedotin, rovalpituzumab tesirine, sacituzumab govitecan, samrotamab vedotin, sirtratumab vedotin, sofituzumab vedotin, taplitumomab paptox, telisotuzumab vedotin, tisotumab vedotin, trastuzumab deruxtecan, trastuzumab duocarmazine, trastuzumab emtansine, tucotuzumab celmoleukin, vadastuximab talirine, vandortuzumab vedotin, and vorsetuzumab mafodotin.

In some embodiments, the immunotherapeutic comprises a drug-to-antibody ratio of at least 5. In some specific embodiments, the immunotherapeutic comprises a drug-to-antibody ratio, which is equal to a sum of instances of the first payload and the second payload in the therapeutically effective amount of the immunotherapeutic divided by instances of the immunotherapeutic in the therapeutically effective amount. In some specific embodiments, the drug-to-antibody ratio is at least 8. In some specific embodiments, the drug-to-antibody ratio is at least 10. Drug-to-antibody ratios for monomeric immunoconjugates range from about 2 to 3 for gemtuzumab ozogamicin (MYLOTARG®) to about 7.6 for sacituzumab govitecan (TRODELVY®) with most FDA-approved therapeutic antibodies having a drug-to-antibody ratio of about 3.5 to 4, including brentuximab vedotin (ADCETRIS®, about 4), trastuzumab emtansine (KADCYLA®, about 3.5), polatuzumab vedotin (POLIVY®, about 3.5): enfortumab vedotin (PADCEV®, about 4), and belantamab mafodotin (BLENREP®, about 4). The immunotherapeutics of this disclosure advantageously carry additional payload relative to monomeric immunoconjugates, which allows for greater delivery of the payload per binding event.

In some embodiments, (1) a combination of equal concentrations of the first immunoconjugate and the second immunoconjugate has a reference IC50, which is a calculated concentration of the first immunoconjugate and the second immunoconjugate necessary to observe 50 percent cytotoxicity in an in vitro assay for a cancer cell line that expresses the antigen; (2) the immunotherapeutic has an improved IC50 against the cancer cell line; and (3) the improved IC50 is less than the reference IC50. In some specific embodiments, (1) a combination of equal concentrations of the first immunoconjugate and the second immunoconjugate has a reference IC50, which is a calculated concentration of the first immunoconjugate and the second immunoconjugate necessary to observe 50 percent cytotoxicity in an in vitro assay for a cancer cell line that expresses the antigen, wherein the calculated concentration is determined based upon the equal concentrations of the first immunoconjugate and second immunoconjugate and not upon a sum of the equal concentrations of the first immunoconjugate and the second immunoconjugate; (2) the immunotherapeutic has an improved IC50 against the cancer cell line; and (3) the improved IC50 is less than the reference IC50.

In some embodiments, the improved IC50 is less than 100 percent of the reference IC50. In some specific embodiments, the improved IC50 is less than 95 percent of the reference IC50. In some very specific embodiments, the improved IC50 is less than 95 percent of the reference IC50.

In some embodiments, (1) the first immunoconjugate has a first reference efficacy against the cancer per mole of the first immunoconjugate, (2) the second immunoconjugate has a second reference efficacy against the cancer per mole of the second immunoconjugate (3) the immunotherapeutic has an improved efficacy against the cancer per mole of the immunotherapeutic; and (4) the improved efficacy is greater than the sum of the first reference efficacy and the second reference efficacy.

In some embodiments, efficacy is determined in vitro, for example, by identifying the viability of cells that are incubated with either the first immunoconjugate, the second immunoconjugate, or the immunotherapeutic.

In some embodiments, efficacy is determined in vivo during pre-clinical research, for example, by identifying the survival rates of animals treated with either the first immunoconjugate, the second immunoconjugate, or the immunotherapeutic, wherein the animals have a cancer that expresses the cancer-associated antigen.

In some embodiments, efficacy is determined in vivo during clinical research, for example, by identifying the survival rates of human subjects treated with either the first immunoconjugate, the second immunoconjugate, or the immunotherapeutic, wherein the human subjects have a cancer that expresses the cancer-associated antigen.

In some embodiments, the improved efficacy is at least 10 percent greater than the sum of the first reference efficacy and the second reference efficacy. In some specific embodiments, the improved efficacy is at least 20 percent greater than the sum of the first reference efficacy and the second reference efficacy. In some very specific embodiments, the improved efficacy is at least 50 percent greater than the sum of the first reference efficacy and the second reference efficacy.

In some embodiments, (1) the first immunoconjugate and the second immunoconjugate have a combined reference efficacy against the cancer per mole of the first immunoconjugate and the second immunoconjugate (2) the immunotherapeutic has an improved efficacy against the cancer per mole of the immunotherapeutic; and (3) the improved efficacy is greater than the combined reference efficacy. The term “combined reference efficacy” refers to the efficacy of a combination of the first immunoconjugate and the second immunoconjugate. For example, (1) the first immunoconjugate and the second immunoconjugate may be combined at the same molarity, and the combination may be used to assess the combined reference efficacy of the combination in vitro or in vivo. The immunotherapeutic may be used at the same molarity to assess the improved efficacy in vitro or in vivo. According to various aspects of the invention, the improved efficacy is greater than the combined efficacy.

In some embodiments, efficacy is determined in vitro, for example, by identifying the viability of cells that are incubated with either (1) both the first immunoconjugate and the second immunoconjugate or (2) the immunotherapeutic.

In some embodiments, efficacy is determined in vivo during pre-clinical research, for example, by identifying the survival rates of animals treated with either (1) both the first immunoconjugate and the second immunoconjugate or (2) the immunotherapeutic, wherein the animals have a cancer that expresses the cancer-associated antigen.

In some embodiments, efficacy is determined in vivo during clinical research, for example, by identifying the survival rates of human subjects treated with either (1) both the first immunoconjugate and the second immunoconjugate or (2) the immunotherapeutic, wherein the human subjects have a cancer that expresses the cancer-associated antigen.

In some embodiments, the improved efficacy is at least 10 percent greater than the combined reference efficacy. In some specific embodiments, the improved efficacy is at least 20 percent greater than the combined reference efficacy. In some very specific embodiments, the improved efficacy is at least 50 percent greater than the combined reference efficacy.

In some embodiments, (1) the first immunoconjugate has a first reference infusion time per mole of the first immunoconjugate; (2) the second immunoconjugate has a second reference infusion time per mole of the second immunoconjugate; (3) the immunotherapeutic has a shorter infusion time per mole of the immunotherapeutic; and (4) the shorter infusion time is less than the sum of the first reference infusion time and the second reference infusion time.

In this specification, the term “infusion time” refers to an amount of time necessary to infuse a therapeutically effective amount of an immunoconjugate or immunotherapeutic of this disclosure to treat a cancer that expresses the cancer-associated antigen in a human.

In some embodiments, the shorter infusion is at least 30 minutes less than the sum of the first reference infusion and the second reference infusion. In some specific embodiments, the shorter infusion is at least 60 minutes less than the sum of the first reference infusion and the second reference infusion.

In some embodiments, the shorter infusion time is shorter than the sum of the first reference infusion and the second reference infusion because the immunotherapeutic has a superior efficacy relative to a combination of the first immunoconjugate and the second immunoconjugate, in which the immunoconjugate and the second immunoconjugate are not crosslinked in the combination. In some specific embodiments, the shorter infusion time is shorter than the reference infusion time because the immunotherapeutic has a superior efficacy per mole relative to the first immunoconjugate and the second immunoconjugate.

In some embodiments, the shorter infusion time is shorter than the reference infusion time because the immunotherapeutic has a greater mass/volume concentration relative to a combination of the first immunoconjugate and the second immunoconjugate, in which the immunoconjugate and the second immunoconjugate are not crosslinked in the combination. Immunotherapeutics of this disclosure may allow for increased mass/volume concentrations relative to monomeric immunotherapeutics, for example, because dimeric immunotherapeutics have molecular weights that are approximately double that of monomeric immunotherapeutics.

In some embodiments, the immunotherapeutic has an infusion time of no greater than 4 hours. In some specific embodiments, the immunotherapeutic has an infusion time of no greater than 3 hours. In some even more specific embodiments, the immunotherapeutic has an infusion time of no greater than 2 hours. In some very specific embodiments, the immunotherapeutic has an infusion time of no greater than 1 hour.

In some embodiments, each antigen-binding site of the first immunoconjugate and the second immunoconjugate comprises (1) a first variable domain that comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7; and (2) a second variable domain that comprises a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10. Dimeric immunoconjugates in which each antigen-binding site comprises the first variable domain and the second variable domain as set forth in this paragraph are generally capable of specifically binding Sp17 with high affinity.

In some embodiments, the first immunoconjugate and the second immunoconjugate each comprise a heavy chain that has at least 90 percent sequence homology with SEQ ID NO: 11 and a light chain that has at least 90 percent sequence homology with SEQ ID NO: 12. Dimeric immunoconjugates that each comprise a heavy chain and a light chain as set forth in this paragraph are generally capable of specifically binding Sp17 with high affinity.

In some embodiments, the immunotherapeutic comprises a dissociation constant (KD) with the cancer-associated antigen of no greater than 25 nanomolar. In some specific embodiments, the immunotherapeutic has a KD with the cancer-associated antigen of no greater than 10 nanomolar. In some very specific embodiments, the immunotherapeutic has a KD with the cancer-associated antigen of no greater than 2.5 nanomolar. In some embodiments, KD is determined by surface plasmon resonance (such as with a Biacore™ instrument).

In some embodiments, the first immunoconjugate and the second immunoconjugate each comprise a human IgG1, IgG2, IgG3, or IgG4 heavy chain constant region and a human kappa or lambda light chain constant region. In some specific embodiments, one or both of the first immunoconjugate and the second immunoconjugate comprise a human IgG1 or IgG4 heavy chain constant region and a human kappa or lambda light chain constant region. In some very specific embodiments, one or both of the first immunoconjugate and the second immunoconjugate comprise a human IgG4 heavy chain constant region and a human kappa light chain constant region.

In some embodiments, one or both of the first immunoconjugate and the second immunoconjugate comprise a IgG2 heavy chain constant region. In some specific embodiments, one or both of the first immunoconjugate and the second immunoconjugate comprise an IgG2a heavy chain constant region.

In some embodiments, one or both of the first immunoconjugate and the second immunoconjugate comprise an IgG4 heavy chain constant region.

In some embodiments, one or both of the first immunoconjugate and the second immunoconjugate comprise a kappa or lambda light chain constant region. In some specific embodiments, one or both of the first immunoconjugate and the second immunoconjugate comprise a kappa light chain constant region.

In some embodiments, one or both of the first immunoconjugate and the second immunoconjugate comprise a human heavy chain constant region and a human light chain constant region. In some specific embodiments, one or both of the first immunoconjugate and the second immunoconjugate comprise a human IgG heavy chain constant region and a human kappa or lambda light chain constant region. In some very specific embodiments, one or both of the first immunoconjugate and the second immunoconjugate comprise a human IgG4 heavy chain constant region and a human kappa light chain constant region.

In some embodiments, the first immunoconjugate and the second immunoconjugate are covalently crosslinked in the immunotherapeutic with an engineered disulfide bond.

In some embodiments, the first immunoconjugate and the second immunoconjugate are covalently crosslinked in the immunotherapeutic with a synthetic linker. In the case of sulfo-SMCC- and SATP-mediated cross-linking, for example, the synthetic linker would be 1-oxo-S—{N-[4-formyl-cyclohexyl(methyl)]-3,4-dihydro-2,5-dioxo-1H-pyrrol-3-yl}-3-thiopropyl, wherein the carbon atom of the formyl is covalently bound to a primary amine of a first of the two IgG antibodies and the 1-carbon atom of the propyl is bound to a primary amine of a second of the two IgG antibodies.

In some embodiments, each antigen-binding site comprises: (1) a first variable domain that comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7; and (2) a second variable domain that comprises a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10.

Various aspects of this disclosure relate to immunotherapeutics that display high-affinity for Sp17. These antibodies were developed using a Fab phage-display bio-panning strategy on a library generated from the peripheral blood of 120 healthy donors as set forth in Example 1 below. The library included about a trillion different combinations. The best-performing Fab in the library had an immunoglobulin heavy chain variable region (VH) with the nucleotide sequence set forth in SEQ ID NO: 1 and an immunoglobulin light chain variable region (VL) with the nucleotide sequence set forth in SEQ ID NO: 2, which nucleotide sequences are depicted in Table 1 below. The VH amino acid sequence is set forth in SEQ ID NO: 3, and the VL amino acid sequence is set forth in SEQ ID NO: 4. The VH and VL amino acid sequences are depicted in Table 2 below, in which CDRs are depicted with bold and underline, and in which framework regions are depicted with plain text. CDR sequences are set forth in SEQ ID NO: 5-10 and are independently depicted in Table 3 below. One of ordinary skill will recognize that the precise demarcation between CDR and framework regions is blurred at least for some of the CDRs, and the CDRs as set forth in Tables 2 & 3 and in SEQ ID NO: 5-10 may therefore include one or more amino acids that might be more-appropriately classified as framework rather than CDR.

Nucleotide sequences encoding the variable regions of the best-performing Fab (SEQ ID NO: 1 & 2) were initially cloned into a mouse IgG2a heavy chain gene and a mouse kappa light chain gene to express a chimeric monoclonal antibody (chAB2). The affinity of the chimeric antibody was determined to be about 2 nanomolar by surface plasmon resonance. Immunohistochemical analysis of the chimeric antibody against 33 normal human tissues indicated that the variable regions lack detectable cross-reactivity.

The human variable regions were then cloned into a human IgG4 heavy chain gene and a human kappa light chain gene to produce a human monoclonal antibody (SP17-AB2) suitable for use as a therapeutic antibody. The heavy chain constant region was mutated to include a Ser228Pro mutation to reduce non-specific interactions with Fc receptor gamma (FcγR). The full heavy chain has the amino acid sequence set forth in SEQ ID NO: 10, and the full light chain has the amino acid sequence set forth in SEQ ID NO: 11, which are depicted in Table 4 below.

Various aspects of this disclosure relate to a pharmaceutical composition comprising an immunotherapeutic as described anywhere in this disclosure and a pharmaceutically acceptable carrier. Suitable pharmaceutically acceptable carriers generally include water with dissolved solutes that buffer pH and provide metal cations and an ionic strength that stabilize an immunotherapeutic of this disclosure. Such formulations are generally sterile, and the selection and preparation of such pharmaceutically acceptable carriers are well known. Solid formats including lyophilized therapeutics generally include, for example, metal cations, anions, and optionally polyols such as sugars (for example, trehalose or glucose) that stabilize the therapeutic in the solid phase and during its reconstitution into an aqueous format. General guidance on selecting pharmaceutically acceptable carriers is available, for example, in REMINGTON: THE SCIENCE AND PRACTICE OF PHARMACY, 22nd edition (Allen Jr, Loyd V., editor) Pharmaceutical Press, 2012, and the skilled practitioner will also look to the formulations of the IgG antibodies described in this disclosure as well as other existing therapeutics in selecting a pharmaceutically acceptable carrier. Such guidance is generally available in the scientific literature and on existing product labels.

In some embodiments, the pharmaceutical composition is suitable for administration to a subject. In some specific embodiments, the pharmaceutical composition is suitable for administration to a human patient. In some very specific embodiments, the pharmaceutical composition is suitable for intravenous administration to a human patient.

Various aspects of this disclosure relate to a kit, comprising (1) a hermetically-sealed container that contains the pharmaceutical composition as described anywhere in this specification and (2) instructions for use of the pharmaceutical composition.

Various aspects of this disclosure relate to a medical device, comprising the pharmaceutical composition as described anywhere in this specification. In some embodiments, the medical device is a syringe, a venous cannula, or a drug-eluting implant.

Various aspects of this disclosure relate to a method of treating or preventing cancer in a subject, comprising identifying that the subject comprises cells that ectopically express the cancer-associated antigen and administering a pharmaceutical composition as described anywhere in this disclosure. Determining that the subject comprises cells that ectopically express the cancer-associated antigen include, for example, identifying mRNA that encodes the cancer-associated antigen by RT-PCR and identifying expression of the cancer-associated antigen by flow cytometry and/or immunohistochemistry. Such methods may advantageously allow determination that a cancer cell expresses the cancer-associated antigen, for example, based upon the prior selection of one or more cancer cells for analysis or based upon the co-identification of expression of the cancer-associated antigen and a cancer phenotype such as the co-expression of the cancer-associated antigen and another cancer antigen.

Various aspects of this disclosure relate to a method to modulate cells that express the cancer-associated antigen in a human subject, comprising administering an immunotherapeutic as described anywhere in this disclosure to the human subject. In some embodiments, the method is a method of treating or preventing cancer in a human subject. In some specific embodiments, the method is a method of treating or preventing cancer in a human subject, the human subject presents with cancer, and at least a portion of the cells that express the cancer-associated antigen are cancer cells.

In some embodiments the immunotherapeutic is administered at an effective amount that is effective to induce cell death in at least a portion of the cells that express the cancer-associated antigen. In some specific embodiments, the immunotherapeutic is conjugated to a radioactive isotope, and the method induces cell death by emitting radiation within or in proximity to the cells that the cancer-associated antigen. In some specific embodiments, the immunotherapeutic is conjugated to a pharmaceutical agent, the pharmaceutical agent is cytotoxic, and the method induces cell death by releasing the pharmaceutical agent within or in proximity to the cells that express the cancer-associated antigen. In some very specific embodiments, the immunotherapeutic is conjugated to a pharmaceutical agent by a labile linker, the pharmaceutical agent is cytotoxic, and the method induces cell death by releasing the pharmaceutical agent within or in proximity to the cells that express the cancer-associated antigen.

In some embodiments, the subject is a mammal. In some specific embodiments, the subject is a rodent, lagomorph, feline, canine, porcine, ovine, caprine, lama, bovine, equine, or primate. In some very specific embodiments, the subject is a human patient.

In some embodiments, the subject is male or female. In some specific embodiments, the subject is female. In some specific embodiments, the subject is male.

In some embodiments, the subject presents with ectopic expression of the cancer-associated antigen. In some specific embodiments, the subject presents with cancer, and cells of the cancer express the cancer-associated antigen.

In some embodiments, the method comprises identifying that the subject comprises cells that express the cancer-associated antigen.

In some embodiments, the method comprises identifying that the subject comprises cells that express the cancer-associated antigen, wherein the cells that express the cancer-associated antigen comprise leukocytes, and the cancer is a lymphoma, leukemia, or myeloma. In some specific embodiments, the method comprises identifying that the subject comprises cells that express the cancer-associated antigen, wherein the cells that express the cancer-associated antigen comprise plasma cells, and the cancer is multiple myeloma. In some specific embodiments, the method comprises identifying that the subject comprises cells that express the cancer-associated antigen, wherein the cells that express the cancer-associated antigen comprise lymphocytes, and the cancer is lymphoma. In some specific embodiments, the method comprises identifying that the subject comprises cells that express the cancer-associated antigen, wherein the cells that express the cancer-associated antigen comprise ovarian cells, and the cancer is ovarian cancer. In some specific embodiments, the method comprises identifying that the subject comprises cells that express the cancer-associated antigen, wherein the cells that express the cancer-associated antigen comprise lung epithelial cells, and the cancer is non-small cell lung cancer.

In some embodiments, the method comprises identifying that a tissue sample of the human subject comprises either RNA encoding the cancer-associated antigen or a protein that includes the cancer-associated antigen prior to the administering.

In some embodiments, the tissue sample is a blood sample.

In some embodiments, the tissue sample is a biopsy. In some specific embodiments, the tissue sample is a tumor biopsy. In some very specific embodiments, the tissue sample is a bone marrow biopsy. In some very specific embodiments, the tissue sample is an ovarian biopsy. In some very specific embodiments, the tissue sample is a lung biopsy.

In some embodiments, the method comprises identifying that the tissue sample comprises a cancer biomarker, wherein the cancer biomarker is neither RNA encoding the cancer-associated antigen nor the cancer-associated antigen.

In some embodiments, the cells that express the cancer-associated antigen comprise leukocytes, and the cancer is a lymphoma, leukemia, or myeloma. In some specific embodiments, the cells that express the cancer-associated antigen comprise leukocytes, and the cancer is multiple myeloma or lymphoma. In some very specific embodiments, the cells that express the cancer-associated antigen comprise plasma cells, and the cancer is multiple myeloma. In some very specific embodiments, the cells that express the cancer-associated antigen comprise lymphocytes, and the cancer is lymphoma.

In some embodiments, the cells that express the cancer-associated antigen comprise ovarian cells, and the cancer is ovarian cancer.

In some embodiments, the cells that express the cancer-associated antigen comprise lung epithelial cells, and the cancer is non-small cell lung cancer.

In some embodiments, the administering is selected from intravenous, intramuscular, subcutaneous, intradermal, intraocular, parenteral, intraperitoneal, intrathecal, intralesional, and intratumoral administration. In some specific embodiments, the administering is intravenous administration.

### EXEMPLIFICATION

### Example 1. Identification of Human Variable Regions for an Anti-Sp17 Antibody Based on Human Diversity

A Fab phage display library was constructed from peripheral blood obtained from 120 healthy human donors. Briefly, this library was constructed by randomly combining nucleotide sequences encoding immunoglobulin heavy chain variable regions with nucleotide sequences encoding immunoglobulin light chain variable regions. The library had a diversity of approximately 1 trillion combinations. Bio-panning of the library identified twelve positive colonies, and nucleotide sequencing of the colonies identified two distinct clones. A single clone was identified as binding Sp17 by ELISA. The nucleotide sequences of the human VH and VL regions of the antibody are set forth in Table 1 below. The variable regions have the amino acid sequences set forth in Table 2 below and the CDRs set forth in Table 3 below.

TABLE 1
Nucleotide sequences of anti-Sp17 VH and VL regions identified by
bio-panning a Fab phage-display library of ~1 trillion human Fabs
developed from peripheral blood samples of 120 human subjects

| SEQ | | |
|---|---|---|
| ID | | Sequence 10 20 30 40 |
| NO. | Region | 12345678901234567890123456789012345678901234567890 |
| 1 | VH | CAGGTACAGCTGCAGCAGTCAGGGGCTGAGGTGAAGAAGCCTGGGTCCTC |
| | | GGTGAAGGTCTCCTGCAAGGCTTCTGGAGGCACCTTCAGCAGCTATGCTA |
| | | TCAGCTGGGTGCGACAGGCCCCTGGACAAGGGCTTGAGTGGATGGGAAGG |
| | | ATCATCCCTATCCTTGGTATAGCAAACTACGCACAGAAGTTCCAGGGCAG |
| | | AGTCACGATTACCGCGGACAAATCCACGAGCACAGCCTACATGGAGCTGA |
| | | GCAGCCTGAGATCTGAGGACACGGCCGTGTATTACTGTGCGAGACCCTCC |
| | | GAAGAGGTGGTAGCTGCTTACGGTGCTTTTGATATCTGGGGCCAAGGGAC |
| | | CACGGTCACCGTCTCAAGC |
| 2 | VL | GAAATTGTGCTGACTCAGTCTCCACTCTCCCTGCCCGTCAGACCTGGGGA |
| | | GCCGGCCTCCATCTCCTGCAGGGCTAGTCAGAGCCTCCTGCGTAGTGACG |
| | | GATTCAACTACTTGGATTGGTACCTGCAGAAGCCAGGGCAGTCTCCACAG |
| | | CTCCTGGTCTATTTGGGTTCTAATCGGGCCTCCGGGGTCCCTGACAGGTT |
| | | CAGTGGCAGTGGATCAGGCACAGATTTTACACTGAAAATCAGCAGAGTGG |
| | | AGGCTGAGGATGTTGGGGTTTATTACTGCATGCAAGCTGTACAAACTCCG |
| | | TACATTTTTGGCCAGGGGACCAAGCTGGAGATCAAA |

TABLE 2
Amino acid sequences of anti-Sp17 VH and
VL regions identified by bio-panning a Fab
phage-display library of ~1 trillion human Fabs
developed from peripheral blood samples
of 120 human subjects

| SEQ ID | | Sequence 10 20 30 40 |
|---|---|---|
| NO. | Region | 12345678901234567890123456789012345678901234567890 |
| 3 | VH | QVQLQQSGAEVKKPGSSVKVSCKASGGTFSSYAISWVRQAPGQGLEWMGR |
| | | IIPILGIANYAQKFQGRVTITADKSTSTAYMELSSLRSEDTAVYYCARPS |
| | | EEVVAAYGAFDIWGQGTTVTVSS |
| 4 | VL | EIVLTQSPLSLPVRPGEPASISCRASQSLLRSDGFNYLDWYLQKPGQSPQ |
| | | LLVYLGSNRASGVPDRFSGSGSGTDFTLKISRVEAEDVGVYYCMQAVQTP |
| | | YIFGQGTKLEIK |

TABLE 3
Amino acid sequences of CDRs of the VH and
VL region sidentified by bio-panning a Fab
phage-display library of ~1 trillion human
Fabs developed from peripheral blood
samples of 120 human subjects

| SEQ ID | | Sequence 10 |
|---|---|---|
| NO. | Region | 12345678901234567 |
| 5 | VH CDR1 | GGTFSSYAIS |
| 6 | VH CDR2 | RIIPILGIANYAQKFQG |
| 7 | VH CDR3 | ARPSEEVVAAYGAFDI |
| 8 | VL CDR1 | RASQSLLRSDGFNYLD |
| 9 | VL CDR2 | LGSNRAS |
| 10 | VL CDR3 | MQAVQTPYIF |

### Example 2. Engineering a Mouse-Human Chimeric Anti-Sp17 IgG2a/KAPPA Monoclonal Antibody

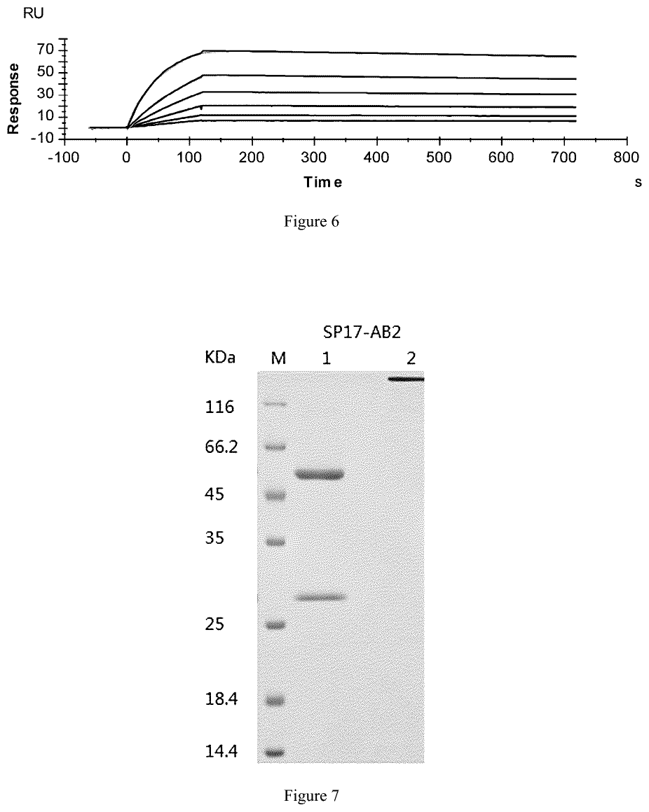

The nucleotide sequences encoding the VH and VL regions of Example 1 were cloned into a mouse IgG2a heavy chain gene and a mouse kappa light chain gene, respectively, and expressed in CHO-S cells to produce a mouse-human chimeric antibody, which was named chAB2. Successful generation of the antibody was confirmed by SDS-PAGE and western blotting using anti-mouse heavy and light chain antibodies and recombinant Sp17 protein (FIGS. 2 & 3). The chAB2 antibody was reactive to mouse surface Sp17 protein expressed on the mouse ovarian cancer cell line ID8 (FIG. 4). HPLC of the chAB2 antibody preparation showed 93 precent purity (FIG. 5). Surface plasmon resonance with a Biacore™ T200 was used to determine a KD for chAB2 and Sp17 of 2.073 nanomolar (FIG. 6).

The specificity of the chAB2 antibody for Sp17 was determined with immunohistochemistry on a normal tissue microarray, which consisted of 33 normal tissues obtained from 2-3 human donors per tissue. The normal tissues included brain, eye, adrenal gland, hypophyseal, thyroid, parathyroid, tonsil, thymus, spleen, heart, lung, larynx, esophagus, stomach, small intestine, colon, liver, pancreas, salivary gland, kidney, bone, skeletal, skin, peripheral nerve, mesothelial, breast, ovary, endometrium, cervix, testis, and prostate. The chAB2 antibody bound to testis and did not bind to normal tissues.

### Example 3. Engineering a Human Anti-Sp17 IgG4/KAPPA Monoclonal Antibody

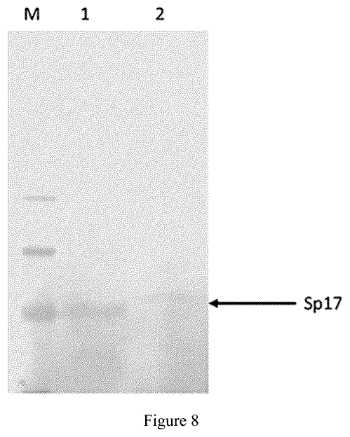

The VH and VL nucleotide sequences of Example 1 were cloned into a human IgG4 heavy chain gene and a human kappa light chain gene, respectively. A Ser228Pro mutation was introduced in the heavy chain to reduce non-specific Fc receptor gamma binding. The resultant antibody was named SP17-AB2. Successful cloning was confirmed by sequence analysis. The amino acid sequences of the heavy chain and light chain are shown in Table 4, and the Ser228Pro mutation is underlined in SEQ ID NO: 11. Successful expression of the SP17-AB2 antibody was confirmed by SDS-PAGE (FIG. 7), and the ability of the SP17-AB2 antibody to bind Sp17 protein was confirmed by western blot (FIG. 8).

TABLE 4
Amino Acid Sequences of the human anti-Sp17 antibody SP17-AB2

| SEQ | | |
|---|---|---|
| ID | | Sequence 10 20 30 40 |
| NO. | Region | 12345678901234567890123456789012345678901234567890 |
| 11 | Full | MDMRVPAQLLGLLLLWLRGARCQVQLQQSGAEVKKPGSSVKVSCKASGGT |
| | Heavy | FSSYAISWVRQAPGQGLEWMGRIIPILGIANYAQKFQGRVTITADKSTST |
| | Chain | AYMELSSLRSEDTAVYYCARPSEEVVAAYGAFDIWGQGTTVTVSSASTKG |
| | | PSVFPLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESKYGPP |
| | | CPPCPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKEYKCKVSN |
| | | KGLPSSIEKTISKAKGQPREPQVYTLPPSQEEMTKNQVSLTCLVKGFYPS |
| | | DIAVEWESNGQPENNYKTTPPVLDSDGSFFLYSRLTVDKSRWQEGNVFSC |
| | | SVMHEALHNHYTQKSLSLSLGK |
| 12 | Full | MDMRVPAQLLGLLLLWLRGARCEIVLTQSPLSLPVRPGEPASISCRASQS |
| | Light | LLRSDGFNYLDWYLQKPGQSPQLLVYLGSNRASGVPDRFSGSGSGTDFTL |
| | Chain | KISRVEAEDVGVYYCMQAVQTPYIFGQGTKLEIKRTVAAPSVFIFPPSDE |
| | | QLKSGTASVVCLLNNFYPREAKVQWKVDNALQSGNSQESVTEQDSKDSTY |
| | | SLSSTLTLSKADYEKHKVYACEVTHQGLSSPVTKSFNRGEC |
| 13 | Signal | MDMRVPAQLLGLLLLWLRGARC |
| | Peptide | |

Based on the foregoing, the SP17-AB2 antibody may be particularly useful as a therapeutic antibody to treat cancers that express Sp17.

### Example 4. Engineering Anti-Sp17 Immunoconjugates with Deruxtecan

The SP17-AB2 antibody as described in Example 3 was crosslinked to deruxtecan using the conjugation strategy set forth in U.S. Pat. No. 10,729,782, which is incorporated by reference in its entirety, to produce an immunoconjugate SP17-AB2-DXD. The deruxtecan pharmaceutical agent displays antineoplastic cytotoxicity based on its exatecan moiety, which is a topoisomerase inhibitor, and which is covalently crosslinked to SP17-AB2 by a linker and a disulfide bond as described in U.S. Pat. No. 10,729,782. Successful production of the immunoconjugate was confirmed by reducing the immunoconjugate with excess dithiothreitol followed by mass spectroscopy analysis to determine that SP17-AB2-DXD comprises a deruxtecan to SP17-AB2 ratio of about 7.93 indicating that, on average, an individual SP17-AB2-DXD molecule comprises about eight deruxtecan moieties. This drug-to-antibody ratio is notably significantly greater than most FDA-approved antibodies.

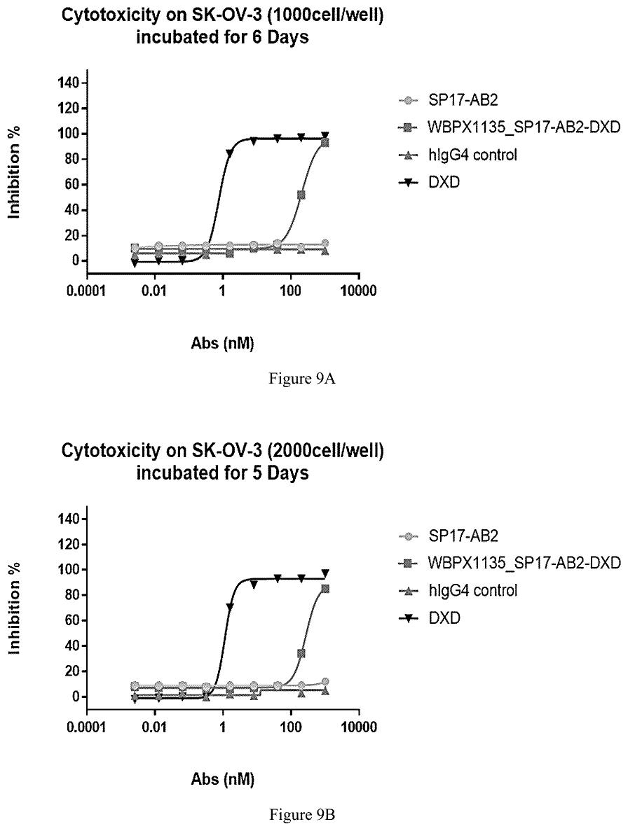

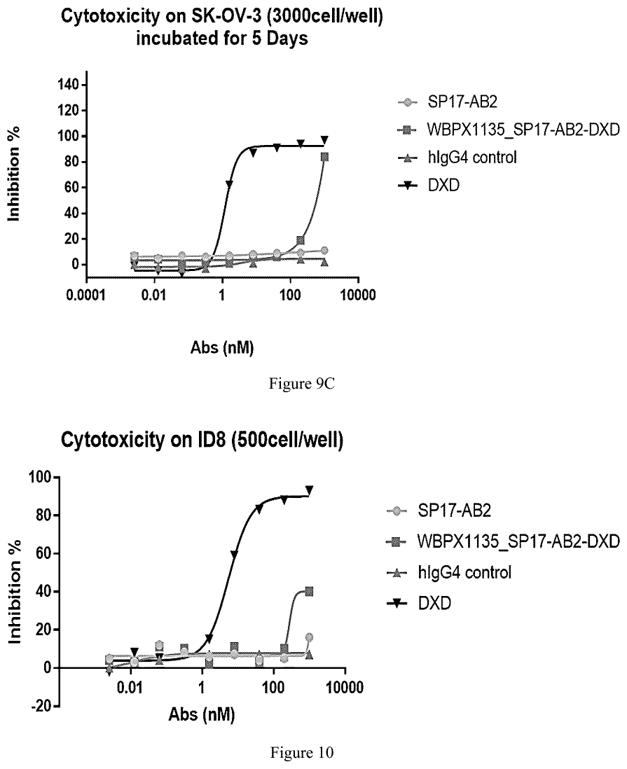

SK-OV-3 cells were seeded at 1000, 2000, or 3000 cells per well and incubated overnight in advance of a cytotoxicity assay. SK-OV-3 cells are human ovarian cancer cells that express Sp17 and that are resistant to various chemotherapeutics including cisplatin, doxorubicin, and diphtheria toxin. The SK-OV-3 cells were treated with various concentrations of the SP17-AB2 antibody, the SP17-AB2-DXD immunoconjugate, a control human IgG4 antibody (hIgG4), or deruxtecan alone (DXD). The cells were incubated for 5 or 6 additional days, and cytotoxicity was then measured using a luminescence assay and En Vision™ plate reader (PerkinElmer, Massachusetts, United States). The SP17-AB2-DXD immunoconjugate displayed superior cytotoxicity relative to the naked SP17-AB2 antibody and the human IgG4 control (FIGS. 9A-9C; Table 5).

TABLE 5
The IC50 and Cytotoxicity of Immunoconjugate
SP17-AB2-DXD are Superior to Naked Antibody
SP17-AB2 Against Human Ovarian Cancer Cells

| | SK-OV-3 cells at | | SK-OV-3 cells at | | SK-OV-3 cells at | |
|---|---|---|---|---|---|---|
| | 1000 cells/well | | 2000 cells/well | | 3000 cells/well | |
| | IC50 | Max Cyto- | IC50 | Max Cyto- | IC50 | Max Cyto- |
| Treatment | (nM) | toxicity | (nM) | toxicity | (nM) | toxicity |
| SP17-AB2 | | 14 | | 12 | | 11 |
| SP17-AB2- | 207 | 93 | 268 | 85 | | 84 |
| DXD | | | | | | |
| hIgG4 | | 10 | | 8 | | 8 |
| control | | | | | | |
| deruxtecan | 0.75 | 98 | 1.1 | 97 | 1.2 | 97 |

ID8 cells were seeded at 250 or 300 cells per well and incubated overnight to establish a confluency of about 90 percent in advance of a second cytotoxicity assay. The cells were then treated with various concentrations of either the SP17-AB2 antibody, the SP17-AB2-DXD immunoconjugate, hIgG4, or deruxtecan alone. The cells were incubated for 3 additional days, and then cytotoxicity was measured. The SP17-AB2-DXD immunoconjugate displayed superior cytotoxicity relative to the naked SP17-AB2 antibody and the human IgG4 control (FIG. 10; Table 6).

TABLE 6
The Cytotoxicity of Immunoconjugate SP17-AB2-DXD is Superior
to Naked Antibody SP17-AB2 Against Mouse Ovarian Cancer Cells

| | ID8 cells at | ID8 cells at |
|---|---|---|
| | 250 cells/well | 500 cells/well |
| Treatment | Max Cytotoxicity | Max Cytotoxicity |
| SP17-AB2 | 29 | 16 |
| SP17-AB2-DXD | 32 | 40 |
| hIgG4 control | 13 | 10 |
| deruxtecan | 94 | 93 |

### Example 5. Engineering Anti-Sp17 Immunoconjugates with Ozogamicin

The SP17-AB2 antibody as described in Example 3 is crosslinked to calicheamicin using the conjugation strategy set forth in U.S. Pat. No. 5,877,296, which is incorporated by reference in its entirety, to produce an immunoconjugate SP17-AB2-Ozogamicin.

### Example 6. Engineering Dimeric Anti-Sp17 Immunoconjugates

1.1 grams of the SP17-AB2-DXD immunoconjugate is adjusted to a concentration of 10 milligrams per milliliter in phosphate-buffered saline (PBS; 0.1 molar phosphate and 0.15 molar sodium chloride at a pH of 7.4) to result in 110 milliliters of a first buffered antibody solution.

10 milligrams of SATP (Pierce Biotechnology, Illinois, United States) is dissolved in 1 milliliter of dimethyl sulfoxide (DMSO) to result in a SATP solution, which is immediately added to the first buffered antibody to produce a SATP reaction. The SATP reaction is stirred for 30 minutes at room temperature to produce a protected immunoconjugate. Excess SATP and DMSO are then removed from the protected immunoconjugate by dialysis into PBS.

1.1 grams of the SP17-AB2-Ozogamicin immunoconjugate is adjusted to a concentration of 10 milligrams per milliliter in PBS to result in 110 milliliters of a second buffered antibody solution.

20 milligrams of sulfo-SMCC (Thermo Scientific, Massachusetts, United States) is dissolved in 4 milliliters of distilled, deionized water to result in a sulfo-SMCC solution, which is immediately added to the second buffered antibody to produce a sulfo-SMCC reaction. The sulfo-SMCC reaction is stirred for 2 hours at 4 degrees Celsius to produce an activated immunoconjugate. Excess sulfo-SMCC is then removed from the activated immunoconjugate by dialysis into PBS.

While the sulfo-SMCC reaction is incubating, 20 milliliters of deacetylation solution is prepared by dissolving 0.7 grams of hydroxylamine HCl and 0.2 grams of ethylenediaminetetraacetate tetrasodium salt (EDTA) in 20 milliliters of PBS and adjusting the pH to 7.2-7.5 with 10 molar aqueous sodium hydroxide. 10 milliliters of the deacetylation solution is then added to the protected immunoconjugate to produce a deprotection solution, which is stirred for two hours at room temperature to produce a sulfhydryl-modified immunoconjugate. Excess hydroxylamine and EDTA are then removed from the sulfhydryl-modified immunoconjugate by dialysis into PBS.

The activated immunoconjugate and the sulfhydryl-modified immunoconjugate are then combined and stirred for 2 hours at 4 degrees Celsius to produce a dimeric immunoconjugate dSP17-AB2-DXD/Ozogamicin, which is purified from side products and monomeric antibodies by size exclusion chromatography.

### Example 7. Dimeric Immunoconjugates Crosslink Cells In Vitro

The monomeric antibody SP17-AB2, the monomeric immunoconjugate SP17-AB2-DXD, the monomeric immunoconjugate SP17-AB2-Ozogamicin, and the dimeric immunoconjugate dSP17-AB2-DXD/Ozogamicin are used to label ID8 cells, and crosslinking of the cells is assessed by flow cytometry. Forward scatter and side scatter indicates that ID8 cells labeled with dSP17-AB2-DXD/Ozogamicin include large populations that are consistent with crosslinked cells. ID8 cells labeled with SP17-AB2, SP17-AB2-DXD, and SP17-AB2-Ozogamicin lack such large populations of cells. This data suggests that dimeric immunoconjugates crosslink cells.

### Example 8. Homodimeric Antibodies Display Greater Cellular Uptake Relative to Monomeric Antibodies

A homodimeric version of SP17-AB2 is prepared according to the strategy set forth in Example 6 and named hdSP17-AB2, which is not conjugated to any pharmaceutical agent. The monomeric antibody SP17-AB2 and the homodimeric antibody hdSP17-AB2 are labeled with fluorescein isothiocyanate (FITC) to produce FITC-SP17-AB2 and FITC-hdSP17-AB2. ID8 cells at various cell densities are incubated with the FITC-labeled antibodies, washed, and then visualized by fluorescence microscopy. The cells incubated with FITC-SP17-AB2 display FITC-associated fluorescence that is localized primarily to the cell peripheries regardless of confluency. Cells at or near 100 percent confluence that are incubated with FITC-hdSP17-AB2 display FITC-associated fluorescence localized between different cells. Cells at less than 50 percent confluence that are incubated with FITC-hdSP17-AB2 display significant FITC-associated fluorescence within the cytosol. These results suggest that homodimeric antibodies display increased cellular uptake relative to monomeric antibodies.

### Example 9. Dimeric Immunoconjugate dSP17-AB2-DXD/Ozogamicin Displays a Lower IC50 and Greater Cytotoxicity Relative to a Combination of Monomeric SP17-AB2-DXD and SP17-AB2-Ozogamicin

SK-OV-3 cells are seeded at 2000 cells per well and incubated overnight in advance of a cytotoxicity assay. The SK-OV-3 cells are then treated with various concentrations of (1) the SP17-AB2 antibody, (2) the SP17-AB2-DXD immunoconjugate, (3) the SP17-AB2-Ozogamicin immunoconjugate, (4) both the SP17-AB2-DXD immunoconjugate and the SP17-AB2-Ozogamicin, (5) the dSP17-AB2-DXD/Ozogamicin immunoconjugate, (6) hdSP17-AB2, (7) hIgG4, or (8) deruxtecan. The cells are incubated for five additional days, and cytotoxicity is then measured using a luminescence assay and En Vision™ plate reader (PerkinElmer, Massachusetts, United States). IC50s and maximum cytotoxicity are then determined.

The IC50 of (4) the combination of SP17-AB2-DXD and SP17-AB2-Ozogamicin is determined based on the concentration of SP17-AB2-DXD and SP17-AB2-Ozogamicin individually and not based on the combined concentration. For example, when SK-OV-3 cells are incubated at a concentration of SP17-AB2-DXD and SP17-AB2-Ozogamicin of 128 nanomolar each, then the IC50 is calculated based on the 128 nanomolar concentration (and not the 256 nanomolar combined concentration) so that 128 nanomolar of (4) SP17-AB2-DXD and SP17-AB2-Ozogamicin contains about the same concentration of deruxtecan, ozogamicin, and antigen-binding sites as 128 nanomolar of (5) the dSP17-AB2-DXD/Ozogamicin immunotherapeutic.

The dimeric immunotherapeutic dSP17-AB2-DXD/Ozogamicin displays both a lower IC50 and greater cytotoxicity than the combination of SP17-AB2-DXD and SP17-AB2-Ozogamicin (Table 7).

TABLE 7
The IC50 & Cytotoxicity of Dimeric Immunoconjugate
dSP17-AB2-DXD/Ozogamicin are Superior to Monomeric
Immunoconjugates and Naked Dimers

| | SK-OV-3 cells at | |
|---|---|---|
| | 2000 cells/well | |
| | IC50 | Max |
| Treatment | (nM) | Cytotoxicity |
| (1) SP17-AB2 | | 10 |
| (2) SP17-AB2-DXD | 270 | 85 |
| (3) SP17-AB2-Ozogamicin | 300 | 80 |
| (4) SP17-AB2-DXD + SP17-AB2-Ozogamicin | 200 | 90 |
| (5) dSP17-AB2-DXD/Ozogamicin | 150 | 95 |
| (6) hdSP17-AB2 | | 15 |
| (7) hIgG4 control | | 10 |
| (8) deruxtecan | 1 | 97 |

What is claimed is:

1. A method to treat cancer in a human subject, comprising administering a therapeutically effective amount of an immunotherapeutic to the subject, wherein:
the immunotherapeutic comprises a first immunoconjugate and a second immunoconjugate that are covalently crosslinked;
the first immunoconjugate comprises a first antibody that binds to a first cancer-associated antigen and a first payload selected from a first radioactive isotope and a first pharmaceutical agent;
the second immunoconjugate comprises a second antibody that binds to a second cancer-associated antigen and a second payload selected from a second radioactive isotope and a second pharmaceutical agent; and
the first payload and the second payload are different.

2. The method as claimed in claim 1, wherein the first antibody and the second antibody have the same amino acid sequence.

3. The method as claimed in claim 1, wherein:
the cancer comprises cells that express human sperm protein 17 (Sp17);
the first antibody and the second antibody each comprise two antigen-binding sites that each specifically bind Sp17 such that the immunotherapeutic comprises exactly four antigen-binding sites that each specifically bind Sp17;
the first antibody and the second antibody each comprise a first variable domain and a second variable;
the first variable domain of the first antibody and the first variable domain of the second antibody have identical amino acid sequences;
the second variable domain of the first antibody and the second variable domain of the second antibody have identical amino acid sequences;
the first variable domain of the first antibody and the second antibody comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7; and
the second variable domain of the first antibody and the second antibody comprises a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10.

4. The method as claimed in claim 1, wherein:
the cancer comprises cells that express human sperm protein 17 (Sp17);
the first antibody and the second antibody each comprise two antigen-binding sites that each specifically bind Sp17 such that the immunotherapeutic comprises exactly four antigen-binding sites that each specifically bind Sp17;
the first antibody comprises a heavy chain that has at least 90 percent sequence homology with SEQ ID NO: 11 and a light chain that has at least 90 percent sequence homology with SEQ ID NO: 12; and
the second antibody comprises a heavy chain that has at least 90 percent sequence homology with SEQ ID NO: 11 and a light chain that has at least 90 percent sequence homology with SEQ ID NO: 12.

5. The method as claimed in claim 1, wherein the first payload is a first radioactive isotope.

6. The method as claimed in claim 5, wherein the first radioactive isotope is an alpha emitter.

7. The method as claimed in claim 5, wherein the first radioactive isotope is selected from actinium-225, astatine-211, bismuth-212, bismuth-213, and terbium-149.

8. The method as claimed in claim 1, wherein the second payload is a second radioactive isotope.

9. The method as claimed in claim 8, wherein the second radioactive isotope is a beta emitter.

10. The method as claimed in claim 8, wherein the second radioactive isotope is selected from copper-67, gallium-68, holmium-166, iodine-124, iodine-131, lutetium-177, samarium-153, technetium-99, and yttrium-90.

11. The method as claimed in claim 1, wherein:
the first payload is a first radioactive isotope;
the first radioactive isotope is an alpha emitter;
the second payload is a second radioactive isotope; and
the second radioactive isotope is a beta emitter.

12. The method as claimed in claim 1, wherein:
the first payload is a first pharmaceutical agent;
the second payload is a second pharmaceutical agent;
the first pharmaceutical agent and the second pharmaceutical agent are selected from a calicheamicin, camptothecin, deruxtecan, doxorubicin, emtansine, exatecan, irinotecan, maleimidocaproyl monomethyl auristatin F, mertansine, monomethyl auristatin F, paclitaxel, PE38, pyrrolobenzodiazepine, SN-38, and vedotin; and
the first pharmaceutical agent and the second pharmaceutical agent are different pharmaceutical agents.

13. The method as claimed in claim 1, wherein:
the immunotherapeutic comprises a drug-to-antibody ratio, which is equal to a sum of instances of the first payload and the second payload in the therapeutically effective amount of the immunotherapeutic divided by instances of the immunotherapeutic in the therapeutically effective amount; and
the drug-to-antibody ratio is at least 5.

14. The method as claimed in claim 1, wherein:
a combination of equal concentrations of the first immunoconjugate and the second immunoconjugate has a reference IC50, which is a calculated concentration of the first immunoconjugate and the second immunoconjugate necessary to observe 50 percent cytotoxicity in an in vitro assay for a cancer cell line that expresses the antigen;
the immunotherapeutic has an improved IC50 against the cancer cell line; and
the improved IC50 is less than the reference IC50.

15. The method as claimed in claim 1, wherein:
the first immunoconjugate has a first reference efficacy against the cancer per mole of the first immunoconjugate;
the second immunoconjugate has a second reference efficacy against the cancer per mole of the second immunoconjugate;
the immunotherapeutic has an improved efficacy against the cancer per mole of the immunotherapeutic; and
the improved efficacy is greater than the sum of the first reference efficacy and the second reference efficacy.

16. The method as claimed in claim 1, wherein:
the first immunoconjugate has a first reference infusion time per mole of the first immunoconjugate;
the second immunoconjugate has a second reference infusion time per mole of the second immunoconjugate;
the immunotherapeutic has a shorter infusion time per mole of the immunotherapeutic; and
the shorter infusion time is less than the sum of the first reference infusion time and the second reference infusion time.

17. An immunotherapeutic, comprising a first immunoconjugate and a second immunoconjugate that are covalently crosslinked, wherein:
the first immunoconjugate comprises a first antibody that binds to a first cancer-associated antigen and a first payload selected from a first radioactive isotope and a first pharmaceutical agent;
the second immunoconjugate comprises a second antibody that binds to a second cancer-associated antigen and a second payload selected from a second radioactive isotope and a second pharmaceutical agent; and
the first payload and the second payload are different.

18. The immunotherapeutic as claimed in claim 17, wherein the first antibody and the second antibody have the same amino acid sequence.

19. The immunotherapeutic as claimed in claim 17, wherein:
the first antibody and the second antibody each comprise two antigen-binding sites that each specifically bind Sp17 such that the immunotherapeutic comprises exactly four antigen-binding sites that each specifically bind Sp17;
the first antibody and the second antibody each comprise a first variable domain and a second variable;
the first variable domain of the first antibody and the first variable domain of the second antibody have identical amino acid sequences;
the second variable domain of the first antibody and the second variable domain of the second antibody have identical amino acid sequences;
the first variable domain of the first antibody and the second antibody comprises a VH CDR1 region comprising an amino acid sequence that is identical to SEQ ID NO: 5, a VH CDR2 region comprising an amino acid sequence that is identical to SEQ ID NO: 6, and a VH CDR3 region comprising an amino acid sequence that is identical to SEQ ID NO: 7; and
the second variable domain of the first antibody and the second antibody comprises a VL CDR1 region comprising an amino acid sequence that is identical to SEQ ID NO: 8, a VL CDR2 region comprising an amino acid sequence that is identical to SEQ ID NO: 9, and a VL CDR3 region comprising an amino acid sequence that is identical to SEQ ID NO: 10.

20. The immunotherapeutic as claimed in claim 17, wherein:
the first antibody and the second antibody each comprise two antigen-binding sites that each specifically bind Sp17 such that the immunotherapeutic comprises exactly four antigen-binding sites that each specifically bind Sp17;
the first antibody comprises a heavy chain that has at least 90 percent sequence homology with SEQ ID NO: 11 and a light chain that has at least 90 percent sequence homology with SEQ ID NO: 12; and
the second antibody comprises a heavy chain that has at least 90 percent sequence homology with SEQ ID NO: 11 and a light chain that has at least 90 percent sequence homology with SEQ ID NO: 12.

21. The immunotherapeutic as claimed in in claim 17, wherein the first payload is a first radioactive isotope.

22. The immunotherapeutic as claimed in claim 21, wherein the first radioactive isotope is an alpha emitter.

23. The immunotherapeutic as claimed in claim 21, wherein the first radioactive isotope is selected from actinium-225, astatine-211, bismuth-212, bismuth-213, and terbium-149.

24. The immunotherapeutic as claimed in claim 17, wherein the second payload is a second radioactive isotope.

25. The immunotherapeutic as claimed in claim 24, wherein the second radioactive isotope is a beta emitter.

26. The immunotherapeutic as claimed in claim 24, wherein the second radioactive isotope is selected from copper-67, gallium-68, holmium-166, iodine-124, iodine-131, lutetium-177, samarium-153, technetium-99, and yttrium-90.

27. The immunotherapeutic as claimed in claim 17, wherein:
the first payload is a first radioactive isotope;
the first radioactive isotope is an alpha emitter;
the second payload is a second radioactive isotope; and
the second radioactive isotope is a beta emitter.

28. The immunotherapeutic as claimed in claim 17, wherein:
the first payload is a first pharmaceutical agent;
the second payload is a second pharmaceutical agent;
the first pharmaceutical agent and the second pharmaceutical agent are selected from a calicheamicin, camptothecin, deruxtecan, doxorubicin, emtansine, exatecan, irinotecan, maleimidocaproyl monomethyl auristatin F, mertansine, monomethyl auristatin F, paclitaxel, PE38, pyrrolobenzodiazepine, SN-38, and vedotin; and
the first pharmaceutical agent and the second pharmaceutical agent are different pharmaceutical agents.

29. The immunotherapeutic as claimed in claim 17, wherein:
a combination of equal concentrations of the first immunoconjugate and the second immunoconjugate has a reference IC50, which is a calculated concentration of the first immunoconjugate and the second immunoconjugate necessary to observe 50 percent cytotoxicity in an in vitro assay for a cancer cell line that expresses the antigen;
the immunotherapeutic has an improved IC50 against the cancer cell line; and
the improved IC50 is less than the reference IC50.

30. The immunotherapeutic as claimed in claim 17, wherein:
the first immunoconjugate has a first reference efficacy against the cancer per mole of the first immunoconjugate;
the second immunoconjugate has a second reference efficacy against the cancer per mole of the second immunoconjugate;
the immunotherapeutic has an improved efficacy against the cancer per mole of the immunotherapeutic; and
the improved efficacy is greater than the sum of the first reference efficacy and the second reference efficacy.

31. The method as claimed in claim 17, wherein:
the first immunoconjugate has a first reference infusion time per mole of the first immunoconjugate;
the second immunoconjugate has a second reference infusion time per mole of the second immunoconjugate;
the immunotherapeutic has a shorter infusion time per mole of the immunotherapeutic; and
the shorter infusion time is less than the sum of the first reference infusion time and the second reference infusion time.


SEQUENCE LISTING (13 sequences)

SEQ ID NO: 1 - DNA, 369 bp, Synthetic construct
  source 1..369: mol_type = other DNA
caggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagaccctccgaagaggtggtagctgcttacggtgcttttgatatctggggccaagggaccacggtcaccgtctcaagc

SEQ ID NO: 2 - DNA, 336 bp, Synthetic construct
  source 1..336: mol_type = other DNA
gaaattgtgctgactcagtctccactctccctgcccgtcagacctggggagccggcctccatctcctgcagggctagtcagagcctcctgcgtagtgacggattcaactacttggattggtacctgcagaagccagggcagtctccacagctcctggtctatttgggttctaatcgggcctccggggtccctgacaggttcagtggcagtggatcaggcacagattttacactgaaaatcagcagagtggaggctgaggatgttggggtttattactgcatgcaagctgtacaaactccgtacatttttggccaggggaccaagctggagatcaaa

SEQ ID NO: 3 - protein, 123 aa, Synthetic construct
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARPSEEVVAAYGAFDIWGQGTTVTVSS

SEQ ID NO: 4 - protein, 112 aa, Synthetic construct
EIVLTQSPLSLPVRPGEPASISCRASQSLLRSDGFNYLDWYLQKPGQSPQLLVYLGSNRASGVPDRFSGSGSGTDFTLKISRVEAEDVGVYYCMQAVQTPYIFGQGTKLEIK

SEQ ID NO: 5 - protein, 10 aa, Synthetic construct
GGTFSSYAIS

SEQ ID NO: 6 - protein, 17 aa, Synthetic construct
RIIPILGIANYAQKFQG

SEQ ID NO: 7 - protein, 16 aa, Synthetic construct
ARPSEEVVAAYGAFDI

SEQ ID NO: 8 - protein, 16 aa, Synthetic construct
RASQSLLRSDGFNYLD

SEQ ID NO: 9 - protein, 7 aa, Synthetic construct
LGSNRAS

SEQ ID NO: 10 - protein, 10 aa, Synthetic construct
MQAVQTPYIF

SEQ ID NO: 11 - protein, 472 aa, Synthetic construct
MDMRVPAQLLGLLLLWLRGARCQVQLQQSGAEVKKPGSSVKVSCKASGGTFSSYAISWVRQAPGQGLEWMGRIIPILGIANYAQKFQGRVTITADKSTSTAYMELSSLRSEDTAVYYCARPSEEVVAAYGAFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 241 aa, Synthetic construct
MDMRVPAQLLGLLLLWLRGARCEIVLTQSPLSLPVRPGEPASISCRASQSLLRSDGFNYLDWYLQKPGQSPQLLVYLGSNRASGVPDRFSGSGSGTDFTLKISRVEAEDVGVYYCMQAVQTPYIFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 22 aa, Synthetic construct
MDMRVPAQLLGLLLLWLRGARC